(19) United States
(12) Patent Application Publication
(10) Pub. No.: US 2010/0233768 A1
(43) Pub. Date: Sep. 16, 2010

(54) METHOD FOR ISOLATING POLYHYROXYALKANOATES

(75) Inventors: Bryan Cooper, Mannheim (DE); Arnold Schneller, Seeheim-Jugenheim (DE); Peter Preishuber-Pflügl, Ludwigshafen (DE)

Correspondence Address: CONNOLLY BOVE LODGE & HUTZ LLP, 1875 EYE STREET, N.W., SUITE 1100, WASHINGTON, DC, 20006, US

(73) Assignee: BASF SE, Ludwigshafen (DE)

(21) Appl. No.: 12/301,782

(22) PCT Filed: May 16, 2007
(86) PCT No.: PCT/EP07/54731

§ 371 Date: Nov. 21, 2008

(30) Foreign Application Priority Data
May 24, 2006 (EP) 06114448.1

(51) Int. Cl. C12P 7/62

(52) U.S. Cl. 435/135

(57) ABSTRACT

The invention relates to a method for isolating polyhydroxyalkanoates from production cells which comprises

i) disintegrating the production cells and subsequently

ii) separating off the cell fragments from the polyhydroxyalkanoate grains by means of a continuous jet separator.

The invention relates to a method for isolating polyhydroxyalkanoates from a production cell.

Polyhydroxyalkanoates (PHAs), such as polyhydroxybutyrates (PHBs) for example, can be synthesized using bacteria. For example, such biotechnological methods are described in Biopolymer, Wiley-VCH, 2002.

PHB occurs at the end of fermentation in the bacterial cells in the form of grains which are surrounded by a protein envelope (J. Biol. Chem. 1989, vol. 264(6), pages 3286-3291). To obtain a sufficiently pure PHB, it must be separated from the bacterial cells.

The biotechnologically produced crude mixtures, in addition to the desired polyhydroxyalkanoate, comprise the microorganisms which have produced the polyhydroxyalkanoate (production cells, biomass, or non-polyhydroxyalkanoate mass). The polyhydroxyalkanoate can be isolated from the biomass a) by dissolving the biomass, b) by extraction of the polyhydroxyalkanoate in a suitable extraction medium or c) by mechanical disintegration of the biomass (production cell) and subsequent separation of the cell fragments from the polyhydroxyalkanoate (PHA)-grains.

The most frequent method for this is extraction of the PHA grains from the biomass using a solvent. As suitable extraction media for polyhydroxyalkanoates, use can be made of chlorinated compounds (method b)). Examples are put forward in EP 0124309 and the literature cited there. The use of solvents has a number of disadvantages as a consequence. One is forced to invest in complex and costly infrastructure for handling and recovering the solvents. The extracted biomass must be freed from the solvent residues before further use as fertilizer or feedstuff. Since PHB dissolves only unsatisfactorily in many solvents, the amounts of solvent which are required are very high.

For breakdown and dissolution of the biomass (workup a)), use can be made of, for example, enzymes or chemical methods. In addition, surface-active compounds can be added. A combination of a plurality of methods is possible.

EP 0145233 describes the breakdown of the biomass by enzymes.

WO 94/24302 describes the breakdown of the biomass by enzymes and hydrogen peroxide.

U.S. Pat. No. 5,110,980 describes the breakdown of the biomass by hypochlorite which makes polyhydroxyalkanoates having a high molecular weight accessible. Different parameters such as temperature, time or pH during the treatment with hypochlorite are studied. Purification of the polyhydroxyalkanoate with dilute acids is not described.

A further method for releasing the polyhydroxybutyrate (PHB) formed from genetically modified Escherichia coli cells has been described (Research In Microbiology 2005, 156, pages 865-873). Here an autolysis step is provided afterwards. The exact autolysis conditions are not described. Autolysis is a process of self-dissolution of the cells by their own enzymes. This preparation method has the following disadvantages. Since the autolysis proceeds incompletely and cell fragments and also PHB grains still adhere to one another, only approximately 80% of the PHB formed is released.

The object is therefore to find a method which leads to complete separation of cell fragments of the production cell from the polyhydroxyalkanoate grains formed.

Since experiments using conventional centrifuges had failed, it was surprisingly found that cell fragments could be very efficiently separated from the polyhydroxyalkanoate grains by means of a continuous jet separator. The pellet comprising the polyhydroxyalkanoate grains is constantly removed by the jets, while the cell fragments are continuously effectively separated off in the overflow (actual “clear runnings”). There is no emptying by opening the drum and as a result also no losses, vortexing and similar instabilities which are detrimental to efficient separation. To achieve still higher purity, the PHA grains can be admixed with clear water and centrifuged again. In this manner an aqueous suspension of PHA grains of high purity is achieved which can then be dried in a known manner, for example by spray drying. The resultant product is suitable for further processing to give thermoplastics. The use of solvents is not necessary.

The method is consequently distinguished from the conventional methods by high efficiency, economic viability and excellent processing ability.

Jet separators are also known under the name Westfalia Separator. A detailed description may be obtained, for example, from www.gea-westfalia.de. By way of example, the VisCon® System may be put forward in which the jets are viscosity controlled. As a result, matching the separator parameters (emptying times) in the case of changed feed conditions is omitted and as a consequence of this, constant solid discharge concentrations are achieved. In the VisCon® system, the jets are not situated on the drum rim, but at a smaller diameter in the drum. Introduction via the hydrohermetic feed and also the outlet via the jets increase the cell activity of the cells separated off.

The necessary equipment is technically available and upscalable as desired, so that the method can be applied without problems to the industrial scale.

A particular embodiment of the inventive method disintegrates the production cells mechanically in step i). The chemical-free disintegration has advantages. It has been described that cells of the PHB-producing bacterium Alcaligenes eutrophus can be disintegrated using a homogenizer (Bioseparation 1991, 2, pages 155-166). The PHB grains present in the cells were virtually completely extracted from the cells after four passages through a homogenizer. The cell suspension, in this type of homogenizer, is pressed through a valve. By adjusting the gap width between valve cone and valve seat, turbulence is generated. The suspension exiting from the valve then impacts a steel plate. The pressure of this machine is therefore restricted to 1500 bar.

To generate higher pressures, large amounts of electrical power are required. Cell disintegration using a homogenizer is economic in particular when the cells are completely disintegrated after a single passage. The method described in Bioseparation 1991, 2, pages 155-166, has the disadvantage that four passages through the homogenizer are required.

We have now surprisingly established that PHA-comprising cells of, in particular, Alcaligenes eutrophus may be very readily disintegrated using a high-pressure homogenizer as described hereinafter. In this case, in a single passage through the high-pressure homogenizer at a pressure of 2000 and more bar, over 99% of the cells are disintegrated and PHA virtually completely released. The present invention therefore also relates to disintegration by means of a high-pressure homogenizer which operates at pressures of 2000 and more atm. For example, it comprises the following arrangement:

Examples of suitable homogenizing devices:

a) comprises an orifice plate having at least one inlet nozzle and an orifice plate having at least one outlet nozzle, in the intermediate space between the orifice plates, if appropriate, mechanical energy being introduced or

b) comprises an orifice plate having at least one inlet nozzle and an impact plate, in the intermediate space between the orifice plate and the impact plate, if appropriate, mechanical energy being introduced.

### Embodiment a)

The homogenizing device for isolating the polyhydroxyalkanoates comprises, for example, an orifice plate having at least one inlet nozzle and an orifice plate having at least one outlet nozzle, the nozzles being arranged axially to one another. In the intermediate space between the orifice plates a static mixer can be situated. If appropriate, in the intermediate space, mechanical energy is additionally introduced.

The orifice plates which can be used according to the inventive method have at least one orifice, that is at least one nozzle. The two orifice plates can each have any desired number of orifices, but preferably no more than in each case 5 orifices, particularly preferably no more than in each case three orifices, very particularly preferably no more than in each case two orifices, and in particular preferably no more than in each case one orifice. Both orifice plates can have a different number or the same number of orifices, preferably both orifice plates have the same number of orifices. Generally, the orifice plates are perforated plates each having at least one orifice.

In another embodiment of this inventive method, the second orifice plate is replaced by a sieve, that is the second orifice plate has a multiplicity of orifices or nozzles. The sieves which can be used can cover a large range of pore sizes, generally the pore sizes are between 0.1 and 250 μm, preferably between 0.2 and 200 μm, particularly preferably between 0.3 and 150 μm, and in particular between 0.5 and 100 μm.

The orifices or nozzles can have any conceivable geometric shape, they can, for example, be circular, oval, polygonal having any desired number of edges, which if appropriate can also be rounded, or else star-shaped. Preferably, the orifices have a circular shape.

The orifices of the inlet orifice plate generally have a diameter of from 0.05 mm to 1 cm, preferably from 0.08 mm to 0.8 mm, particularly preferably from 0.1 to 0.5 mm, and in particular from 0.2 to 0.4 mm. The orifices of the outlet orifice plate generally have a diameter of from 0.5 mm to 1 cm, preferably from 5 mm to 50 mm, particularly preferably from 10 to 20 mm.

The two orifice plates are preferably constructed in such a manner that the orifices or nozzles are arranged axially to one another. Axial arrangement is to be taken to mean that the flow direction generated by the geometry of the nozzle orifice is identical for the two orifice plates. The orifice directions of the inlet nozzle and outlet nozzle for this need not lie on a line, they can also be displaced in parallel, as follows from the above statements. Preferably, the orifice plates are directed in parallel.

However, other geometries are possible, in particular non-parallel orifice plates, or different orifice directions of the inlet and outlet nozzles. In the two-orifice-plate system (inlet orifice plate and outlet orifice plate), as set forth above, the outlet nozzle has larger orifices. As a result, the turbulence is calmed. An impact plate is not necessary in this case.

The thickness of the orifice plates can be as desired. Preferably, the orifice plates have a thickness in the range of from 0.1 to 100 mm, preferably from 0.5 to 30 mm, and particularly preferably from 1 to 10 mm. The thickness (I) of the orifice plates is selected in such a manner that the quotient of diameter (d) of the orifices and thickness (I) is in the range of 1:1, preferably 1:1.5, and particularly preferably 1:2.

The intermediate space between the two orifice plates can be as long as desired, generally the length of the intermediate space is 1 to 500 mm, preferably 10 to 300 mm, and particularly preferably 20 to 100 mm.

In the intermediate space between the orifice plates, a static mixer can be situated which can completely or partially fill up the section between the two orifice plates. Preferably, the static mixer extends over the entire length of the intermediate space between the two orifice plates. Static mixers are known to those skilled in the art. A static mixer can be, for example, a valve mixer, or a static mixer having boreholes, one made of fluted lamellae, or one made of engaging ribs. In addition, it can be a static mixer in spiral shape or in an N shape, or one having heatable or coolable mixing elements.

In addition to the static mixer, in the intermediate space between the two orifice plates, mechanical energy can be introduced. The energy can be introduced, for example in the form of mechanical vibrations, ultrasound or rotational energy. As a result, a turbulent flow is produced which has the effect that the particles do not agglomerate in the intermediate space.

### Embodiment b)

Alternatively to this first variant, the mixing device can comprise an orifice plate having at least one inlet nozzle and an impact plate, in the intermediate space between the orifice plate and the impact plate, if appropriate, a static mixer being situated. Alternatively, or in addition to the static mixer, mechanical energy can be introduced in the intermediate space.

The aforesaid applies to the orifice plate having inlet nozzle, the intermediate space having a static mixer and mechanical energy introduction.

In this variant, the second orifice plate is replaced by an impact plate. The impact plate generally has a diameter which is 0.5 to 20%, preferably 1 to 10%, smaller than the tubular diameter at the point at which the impact plate is installed.

In general, the impact plate can have any geometrical shape, preferably in the form of a round disk, so that, in frontal view, a ring gap may be seen. The form of a slot or a channel, for example, is also conceivable.

The impact plate, in a similar manner to the second orifice plate in the abovedescribed variant, can be affixed at different distances with respect to the first orifice plate. As a result, the intermediate space between the orifice plate and the impact plate can be of any desired length; generally, the length of the intermediate space is 1 to 500 mm, preferably 10 to 300 mm, and particularly preferably 20 to 100 mm.

The inventive method has some advantages over the methods known from the prior art, since particularly high yields of the polyhydroxyalkanoate of high molecular weight are obtained. In particular, polyhydroxyalkanoates having Mn of 50 000 to 2 000 000, and in particular from 100 000 to 200 000, may be achieved by this workup variant.

The temperature at which the crude emulsion is emulsified to give the finely divided emulsion by the inventive method is generally 0 to 150° C., preferably 5 to 80° C., particularly preferably 20 to 40° C. In this case all of the homogenizing units used in the device can be heated/cooled.

The homogenization is generally carried out at pressures above atmospheric pressure, that is >1 bar. In this case, however, the pressures do not exceed a value of 10 000 bar, so that preferably homogenization pressures of >1 bar to 10 000 bar, preferably 5 to 2500 bar, and particularly preferably from 100 to 2000 bar, are established.

The production cell concentrations used in the inventive method are about 20 to 300 g/l, preferably 50-220 g/l.

Any type of cell or cell layer in this case is termed production cell; in particular those cells of animal, plant or microbial origin. Equally preferably, production cells are recombinant organisms. Particularly highly suitable production cells are prokaryotes (including the Archaea) or eukaryotes, particularly bacteria, including halobacteria and methanococci, fungi, insect cells, plant cells and mammal cells, particularly preferably Alcaligenes eutrophus, Escherichia coli, Bacillus subtilis, Bacillus megaterium, Aspergillus oryzea, Aspergillus nidulans, Aspergillus niger, Pichia pastoris, Pseudomonas spec., Lactobacillen, Hansenula polymorpha, Trichoderma reesei, SF9 (or related cells). Particularly preferably, the microorganism is Alcaligenes eutrophus.

The production cell can be used in the inventive method directly after culturing (e.g. fermentation); but it is also possible first to kill the production cell, for example by sterilization, and if appropriate to enrich the cell mass by filtration of the culture medium.

Polyhydroxyalkanoates are taken to mean biotechnologically produced polymers. In particular, these are taken to mean the following: poly(3-hydroxybutyrate) (P-3HB), poly(3-hydroxybutyrate)/co-3-hydroxyvalerate (P-3HBco-3HV), poly(3-hydroxybutyrate)/co-4-hydroxybutyrate (P-3HB-co-4HB), poly(3-hydroxybutyrate)/co-3-hydroxyhexanoate (P-3HB-co-3HHx) and poly(3-hydroxybutyrate)/co-3-hydroxyoctanoate (P-3HB-co-3HO).

Equipment used:

In the example, as high-pressure homogenizer for disintegrating the production cells, the following arrangement I was selected. As inlet nozzle, use was made of an orifice plate having 14×0.2 mm wide boreholes. The fermentation broth was a suspension and was forced through the orifice plate at a pressure of approximately 2000 atm. In the intermediate space (15 mm long and 8 mm in diameter), the suspension was vortexed before it encountered the second orifice plate which acted as outlet nozzle. The cell suspension was passed through a conical borehole to the outlet orifice plate and then exited from the orifice plate block from a single borehole (diameter 1.5 mm). The outlet orifice plate was centrally arranged compared with the boreholes of the inlet nozzle.

As jet separator, use was made of an instrument from the company GEA Westfalia type HFC-15.

### EXAMPLE 1

### Isolation of 3-hydroxypolyhydroxybutyrate (3-PHB) from Alcaligenes eutrophus Production Cells

i) Fermentation of 3-hydroxypolyhydroxybutyrate in Alcaligenes eutrophus production cells:
The fermentation was performed according to Kim, Lee, Lee, Chang, Chang and Woo in Biotechnology and Bioengineering, vol. 43, pages 892-898 (1994).

ii) Disintegration of the cells and separation of the 3-PHB grains:
3300 liters of Alcaligenes eutrophus fermentation broth having a content of 90 g/l of biodry mass, thereof of 80% PHB, after completion of fermentation, were cooled to 2° C. in the fermenter. The broth is then passed through a high-pressure homogenizer I at 2000 bar pressure. Since the pressure first had to build up, the first liters of broth were collected separately and recirculated to the fermenter. The fermentation broth was passed in entirety through the high-pressure homogenizer.
The effectiveness of cell disintegration was measured by plating the fermentation broth onto a suitable nutrient agar before and after disintegration. The viable cell count before disintegration was 5×10¹⁰ cfu/ml (=100%). After cell disintegration, the viable cell count was determined in the same manner. It was 5×10⁶ cfu/ml. This corresponds to an effectiveness of high-pressure disintegration of 99.99%.
The cell homogenate was then passed through a jet separator type HFC-15 from GEA Westfalia. The material which was deposited by centrifugal force (concentrate) was collected separately from the cell debris which was not deposited (overflow). Total dry matter and PHB concentration were each determined (see table below for results). The concentrate was diluted with demineralized water to the original starting volume and again centrifuged. The process was repeated once more.

iii) Drying the 3-PHB grains
The 3-PHB suspension obtained from ii) was spray dried in a conventional spray drier. The drying gas was nitrogen having a gas inlet temperature of 200° C., gas outlet temperature 90° C. The PHB suspension was atomized using a two-fluid nozzle. The dry product was discharged from the gas stream via a star wheel lock. The experimental results are listed in the table below.

| | | Concentration | Total mass dry | Concentration | Total mass | 3-PHB |
|---|---|---|---|---|---|---|
| | | dry matter | matter | 3-PHB | 3-PHB | fraction |
| Fraction | Total mass | [g/kg] | [kg] | [g/kg] | [kg] | [g/g] |
| Fermentation broth | 3300 | 80 | 264 | 64 | 211 | 0.800 |
| Homogenate | 3400 | 77.6 | 264 | 62.1 | 211 | 0.800 |
| 1st concentrate | 780 | 284.2 | 221.7 | 270 | 211 | 0.950 |
| 1st overflow | 2620 | 16.15 | 42.3 | 0 | 0 | 0.000 |
| 2nd concentrate | 770 | 276.8 | 213.1 | 274 | 211 | 0.990 |
| 2nd overflow | 2630 | 3.3 | 8.6 | 0 | 0 | 0.000 |
| 3rd concentrate | 760 | 277.6 | 211 | 277.5 | 210.9 | 0.999 |
| 3rd overflow | 2640 | 0.80 | 2.1 | 0 | 0 | 0.000 |
| Spray drier | 215.2 | 980 | 210.9 | 980 | 210.9 | 0.999 |

1. A method for isolating polyhydroxyalkanoates from production cells which comprises
i) disintegrating the production cells and subsequently
ii) separating off the cell fragments from the polyhydroxyalkanoate grains by means of a continuous jet separator.

2. The method according to claim 1, wherein the production cells are disintegrated in step i) by means of a high-pressure homogenizing device.

3. The method according to claim 2, wherein the homogenizing device
a) comprises an orifice plate having at least one inlet nozzle and an orifice plate having at least one outlet nozzle, in the intermediate space between the orifice plates, if appropriate, mechanical energy being additionally introduced or
b) comprises an orifice plate having at least one inlet nozzle and an impact plate, in the intermediate space between the orifice plate and the impact plate, if appropriate, mechanical energy being introduced.

4. The method according to claim 1, wherein the production cell is a recombinant organism.

5. The method according to claim 1, wherein the polyhydroxyalkanoate is a poly(3-hydroxybutyrate) (P-3HB), poly(3-hydroxybutyrate)/co-3-hydroxyvalerate (P-3HB-co-3HV), poly(3-hydroxybutyrate)/co-4-hydroxybutyrate (P-3HB-co-4HB), poly(3-hydroxybutyrate)/co-3-hydroxyhexanoate (P-3HB-co-3HHx) or poly(3-hydroxybutyrate)/co-3-hydroxyoctanoate (P-3HB-co-3HO).

6. The method according to claim 1, wherein the production cell is killed before homogenization.
